(19) United States
(12) Patent Application Publication
(10) Pub. No.: US 2025/0242147 A1
(43) Pub. Date: Jul. 31, 2025

(54) CONTROL ARCHITECTURE FOR A MEDICAL DEVICE

(71) Applicant: Abiomed, Inc., Danvers, MA (US)

(72) Inventors: Hisham Hafez, Danvers, MA (US); Martin Kortyka, Danvers, MA (US)

(73) Assignee: Abiomed, Inc., Danvers, MA (US)

(21) Appl. No.: 19/036,456

(22) Filed: Jan. 24, 2025

Related U.S. Application Data
(60) Provisional application No. 63/625,785, filed on Jan. 26, 2024.

(51) Int. Cl. A61M 60/508; A61M 60/13; A61M 60/216; A61M 60/585; A61M 60/592; H04L 67/12

(52) U.S. Cl. CPC A61M 60/508; A61M 60/13; A61M 60/216; A61M 60/585; A61M 60/592; H04L 67/12; A61M 2205/18; A61M 2205/3584

(57) ABSTRACT

Methods and apparatus for controlling a medical device are provided. A controller may include a first computing device configured to control the medical device, a second computing device configured to connect to at least one network device, and a processor configured to selectively enable or disable a switchable data communication network coupled between the first computing device and the second computing device.

### RELATED APPLICATIONS

This application claims the benefit under 35 U.S.C. § 119 (e) to U.S. Provisional Patent Application No. 63/625,785, filed Jan. 26, 2024, and titled, “CONTROL ARCHITECTURE FOR A MEDICAL DEVICE,” the entire contents of which is incorporated by reference herein.

### TECHNICAL FIELD

This disclosure relates to a control architecture for a medical device.

### BACKGROUND

Medical devices often include a controller configured to control operation of the device. Temporary cardiac support devices, such as heart pump systems (also referred to as “intracardiac blood pumps”) are a type of medical device that provides hemodynamic support and facilitate heart recovery. Intracardiac blood pumps have traditionally been used to temporarily assist the pumping function of a patient's heart during emergent cardiac procedures, such as a stent placement, performed after the patient suffers a heart attack, cardiac arrest, and/or cardiogenic shock. Intracardiac blood pumps also may be used to take the load off of a patient's heart to allow the heart to recover from such a cardiac procedure or from a heart attack, cardiac arrest, cardiogenic shock, or heart damage (e.g., caused by a viral infection). In that regard, an intracardiac blood pump can be introduced into the heart either surgically or percutaneously and used to deliver blood from one location in the heart or circulatory system to another location in the heart or circulatory system. For example, when deployed in the left heart, an intracardiac blood pump can pump blood from the left ventricle of the heart into the aorta. Likewise, when deployed in the right heart, an intracardiac blood pump can pump blood from the inferior vena cava into the pulmonary artery. Intracardiac pumps can be powered by a motor located outside of the patient's body via an elongate drive shaft (or drive cable) or by an onboard motor located inside the patient's body. Examples of such devices include the Impella® family of devices (Abiomed, Inc., Danvers, MA).

### SUMMARY

Described herein is a control architecture for a medical device, such as a cardiac support system. The control architecture may include a first computing device configured to receive data from one or more medical devices (e.g., a right heart pump and a left heart pump) and control operation of the one or more medical devices, and a second computing device configured to communicate with one or more network-connected devices (e.g., one or more cloud-based resources). The control architecture may include an onboard switchable data communication network coupled between the first computing device and the second computing device. A processor may be configured to selectively enable or disable the switchable data communication network thereby permitting or disallowing data transfer (e.g., high speed data transfer) between the first computing device and the second computing device. Use of an onboard switchable data communication network may facilitate on the one hand maintaining separation between processes (e.g., control software) executing on the first computing device during operation of the medical device and processes (e.g., data analysis software) executing on the second device during operation of the device and on the other hand, allowing for onboard parallel processing when desired without compromising patient safety.

In some embodiments, a controller for a medical device is provided. The controller includes a first computing device configured to control the medical device, a second computing device configured to connect to at least one network device, wherein the first computing device and the second computing device are coupled via a switchable data communication network, and a processor configured to selectively enable or disable the switchable data communication network.

In one aspect, the controller further includes a firewall component arranged between the first computing device and the second computing device, the firewall component configured to filter communication between the first computing device and the second computing device. In another aspect, the controller further includes a first switch, and the processor is further configured enable or disable the first switch to direct at least some communication between the first computing device and the second computing device through the firewall component. In another aspect, the controller further includes a video conversion component arranged between the first computing device and the second computing device, the video conversion component being configured to convert a first video stream received from the first computing device and having first format to a second video stream having a second format. In another aspect, the controller further includes a second switch. The processor is further configured to control a state of the second switch, the second switch is in a first state, the first video stream is provided to an external device, and when the second switch is in a second state, the first video stream is converted to the second video stream, the second video stream being provided as input to the second computing device.

In another aspect, the second computing device includes at least one user interface. In another aspect, the at least one user interface includes a switchable interface. In another aspect, the processor is further configured to determine when a connector having a particular configuration is connected to the at least one user interface, and disable the switchable interface when it is determined that a connector having the particular configuration is connected to the at least one user interface. In another aspect, the first computing device is a first system on chip module (SOM) and the second computing device is a second SOM. In another aspect, the switchable data communication network is an Ethernet network. In another aspect, the processor is configured to selectively enable the switchable data communication network when the second computing device is disconnected from the at least one network device. In another aspect, the processor is configured to selectively disable the switchable data communication network when the second computing device is connected to the at least one network device. In another aspect, the processor is configured to selectively enable or disable the switchable data communication network based, at least in part, on an operating state of the medical device. In another aspect, the processor is configured to selectively disable the switchable data communication network when the medical device is operating.

In another aspect, the controller further includes a storage device in communication with the processor, wherein the processor is further configured to store a plurality of states associated with switches in the controller. In another aspect, the processor is further configured to selectively provide power to one or more components of the controller. In another aspect, the controller further includes fault detection circuitry arranged to detect a fault or a reset condition of the processor. In another aspect, the fault detection circuitry is configured to output an alarm signal in response to detecting a fault or a reset condition of the processor. In another aspect, the processor is configured to suppress output of the alarm signal unless a fault or a reset condition of the processor occurs. In another aspect, the first computing device, the second computing device and the processor are arranged on a printed circuit board.

In some embodiments, a method of controlling a medical device is provided. The method includes sending control signals from a first computing device to control an operation of the medical device, receiving, by the first computing device, data from the medical device, and selectively enabling or disabling, by a processor, a switchable data communication network, the switchable data communication network arranged to enable communication between the first computing device and a second computing device configured to connect to at least one network device.

In one aspect, the method further includes filtering at least some communication between the first computing device and the second computing device using a firewall component. In another aspect, the firewall component includes a first switch and the method further include selectively enabling or disabling the first switch to direct the at least some communication between the first computing device and the second computing device through the firewall component. In another aspect, the method further includes converting, using a video conversion component arranged between the first computing device and the second computing device, a first video stream received from the first computing device and having first format to a second video stream having a second format. In another aspect, the video conversion component includes a second switch and the method further includes when the second switch is in a first state, providing the first video stream to an external device, and when the second switch is in a second state, converting the first video stream to the second video stream, and providing the second video stream as input to the second computing device.

In another aspect, the method further includes determining when a connector having a particular configuration is connected to a user interface of the second computing device, and disabling a switchable interface of the user interface when it is determined that a connector having the particular configuration is connected to the user interface. In another aspect, the first computing device is a first system on chip module (SOM) and the second computing device is a second SOM. In another aspect, the switchable data communication network is an Ethernet network.

In another aspect, selectively enabling or disabling the switchable data communication network comprises selectively enabling the switchable data communication network when the second computing device is disconnected from the at least one network device. In another aspect, selectively enabling or disabling the switchable data communication network comprises selectively disabling the switchable data communication network when the second computing device is connected to the at least one network device. In another aspect, selectively enabling or disabling the switchable data communication network comprises selectively enabling or disabling the switchable data communication network based, at least in part, on an operating state of the medical device. In another aspect, selectively enabling or disabling the switchable data communication network based, at least in part, on an operating state of the medical device comprises selectively disabling the switchable data communication network when the medical device is operating.

In another aspect, the method further includes storing, on a storage device in communication with the processor, a plurality of states associated with switches in a controller that includes the first computing device and the second computing device. In another aspect, the method further includes selectively providing power to one or more components of a controller that includes the first computing device and the second computing device. In another aspect, the method further includes detecting, using fault detection circuitry, a fault or a reset condition of the processor. In another aspect, the method further includes outputting, by the fault detection circuitry, an alarm signal in response to detecting a fault or a reset condition of the processor. In another aspect, the method further includes suppressing, by the processor, output of the alarm signal unless a fault or a reset condition of the processor occurs.

In some embodiments, a printed circuit board is provided. The printed circuit board includes a first system on a chip module (SOM) configured to control a medical device, a second SOM configured to connect to at least one network device, and a switchable data communication network coupled between the first SOM and the second SOM.

In one aspect, the printed circuit board further includes a firewall component arranged between the first SOM and the second SOM, the firewall component configured to filter communication between the first SOM and the second SOM. In another aspect, the printed circuit board further includes a first switch and a processor, wherein the processor is further configured enable or disable the first switch to direct at least some communication between the first SOM and the second SOM through the firewall component. In another aspect, the printed circuit board further includes a video conversion component arranged between the first SOM and the second SOM. The video conversion component is configured to convert a first video stream received from the first SOM and having first format to a second video stream having a second format. In another aspect, the printed circuit board further includes a second switch and a processor. The processor is configured to control a state of the second switch such that when the second switch is in a first state, the first video stream is provided to an external device, and when the second switch is in a second state, the first video stream is converted to the second video stream, the second video stream being provided as input to the second SOM.

In another aspect, the second SOM includes at least one user interface. In another aspect, the at least one user interface includes a switchable interface. In another aspect, the printed circuit board includes a processor, wherein the processor is configured to determine when a connector having a particular configuration is connected to the at least one user interface, and disable the switchable interface when it is determined that a connector having the particular configuration is connected to the at least one user interface. In another aspect, the switchable data communication network is an Ethernet network. In another aspect, the printed circuit board further includes a processor configured to selectively enable the switchable data communication network when the second SOM is disconnected from the at least one network device. In another aspect, the printed circuit board further includes a processor configured to selectively disable the switchable data communication network when the second SOM is connected to the at least one network device. In another aspect, the printed circuit board further includes a processor configured to selectively enable or disable the switchable data communication network based, at least in part, on an operating state of the medical device. In another aspect, the processor is configured to selectively disable the switchable data communication network when the medical device is operating. In another aspect, the printed circuit board further includes a processor and a storage device in communication with the processor, wherein the processor is configured to store a plurality of states associated with switches located on the printed circuit board.

In another aspect, the printed circuit board further includes a processor configured to selectively provide power to one or more components on the printed circuit board. In another aspect, the printed circuit board further includes a processor and fault detection circuitry arranged to detect a fault or a reset condition of the processor. In another aspect, the fault detection circuitry is configured to output an alarm signal in response to detecting a fault or a reset condition of the processor. In another aspect, the processor is configured to suppress output of the alarm signal unless a fault or a reset condition of the processor occurs.

### BRIEF DESCRIPTION OF DRAWINGS

FIG. 1 shows an illustrative heart pump device that may be controlled using a controller, in accordance with some embodiments.

FIG. 2 is a block diagram of a first example controller architecture, in accordance with some embodiments.

FIG. 3 is a block diagram of a second example controller architecture, in accordance with some embodiments.

FIG. 4 is a schematic of a switching architecture of a controller, in accordance with some embodiments.

FIG. 5 is a schematic showing example communication paths between a first computing device and a second computing device of a controller, in accordance with some embodiments.

FIG. 6 is a schematic showing an architecture for controlling switches of a controller, in accordance with some embodiments.

FIG. 7 is a flowchart of a process for selectively enabling or disabling an internal data communication network of a controller, in accordance with some embodiments.

FIG. 8 is a flowchart of a process for monitoring a processor for a fault or reset condition, in accordance with some embodiments.

FIG. 9 is a schematic of a printed circuit board including multiple computing devices and a standard edge connector for an expansion board, in accordance with some embodiments.

### DETAILED DESCRIPTION

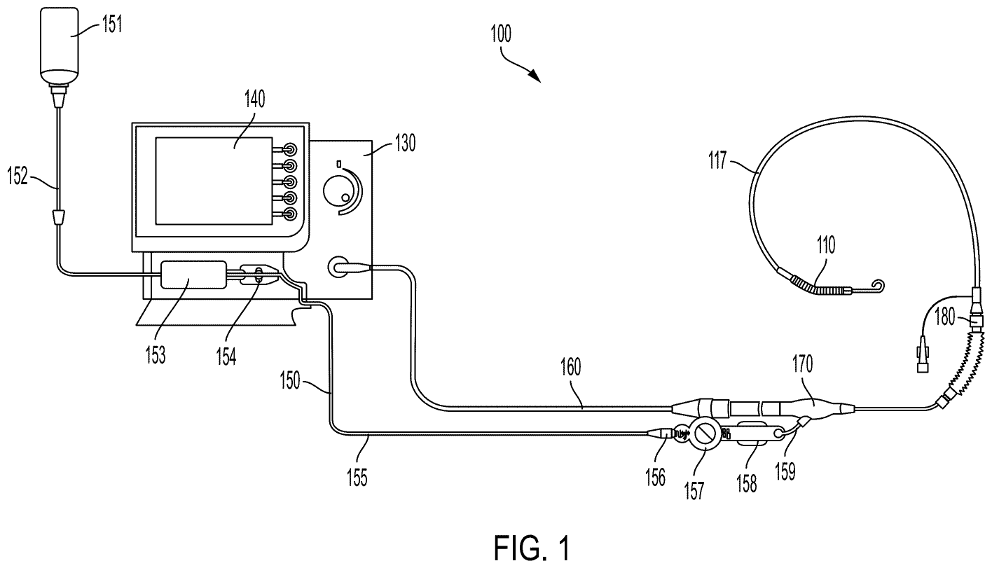

A circulatory support device (also referred to herein as a “heart pump” or simply a “pump”) may include a percutaneous, catheter-based device that provides hemodynamic support to the heart of a patient. As shown in FIG. 1, heart pump 110 may form part of a cardiac support system 100. Cardiac support system 100 also may include a controller 130 (e.g., an Automated Impella Controller®, referred to herein as an “AIC,” from ABIOMED, Inc., Danvers, Mass.), a display 140, a purge subsystem 150, a connector cable 160, a plug 170, and a repositioning unit 180. As shown, controller 130 may include display 140. Controller 130 may be configured to monitor and control operation of heart pump 110. During operation, purge subsystem 150 may be configured to deliver a purge fluid to heart pump 110 through catheter tube 117 to prevent blood from entering the motor (not shown) of the heart pump. In some implementations, the purge fluid is a dextrose solution (e.g., 5% dextrose in water with 25 or 50 IU/mL of heparin, although the solution need not include heparin in all embodiments). Connector cable 160 may provide an electrical connection between heart pump 110 and controller 130. Plug 170 may connect catheter tube 117, purge subsystem 150, and connector cable 160. In some implementations, plug 170 may include a storage device (e.g., a memory) configured to store, for example, operating parameters to facilitate transfer of the patient to another controller if needed. Repositioning unit 180 may be used to reposition heart pump 110 in the patient's heart.

As shown in FIG. 1, in some embodiments, the cardiac support system 100 may include a purge subsystem 150 having a container 151, a supply line 152, a purge cassette 153, a purge disc 154, purge tubing 155, a check valve 156, a pressure reservoir 157, an infusion filter 158, and a sidearm 159. Container 151 may, for example, be a bag or a bottle. As will be appreciated, in other embodiments the cardiac support system 100 may not include a purge subsystem. In some embodiments, a purge fluid may be stored in container 151. Supply line 152 may provide a fluidic connection between container 151 and purge cassette 153. Purge cassette 153 may control how the purge fluid in container 151 is delivered to heart pump 110. For example, purge cassette 153 may include one or more valves for controlling a pressure and/or flow rate of the purge fluid. Purge disc 154 may include one or more pressure and/or flow sensors for measuring a pressure and/or flow rate of the purge fluid. As shown, controller 130 may include purge cassette 153 and purge disc 154. Purge tubing 155 may provide a fluidic connection between purge disc 154 and check valve 156. Pressure reservoir 157 may provide additional filling volume during a purge fluid change. In some implementations, pressure reservoir 157 includes a flexible rubber diaphragm that provides the additional filling volume by means of an expansion chamber. Infusion filter 158 may help prevent bacterial contamination and air from entering catheter tube 117. Sidearm 159 may provide a fluidic connection between infusion filter 158 and plug 170. Although shown as having separate purge tubing and connector cable, it will be appreciated that in some embodiments, the cardiac support system 100 may include a single connector with both fluidic and electric lines connectable to the controller 130.

During operation, controller 130 may be configured to receive measurements from one or more pressure sensors (not shown) included as a portion of heart pump 110 and purge disc 154. Controller 130 may also be configured to control operation of the motor (not shown) of the heart pump 110 and purge cassette 153. As noted herein, controller 130 may be configured to control and measure a pressure and/or flow rate of a purge fluid via purge cassette 153 and purge disc 154. During operation, after exiting purge subsystem 150 through sidearm 159, the purge fluid may be channeled through purge lumens (not shown) within catheter tube 117 and plug 170. Sensor cables (not shown) within catheter tube 117, connector cable 160, and plug 170 may provide an electrical connection between components of the heart pump 110 (e.g., one or more pressure sensors) and controller 130. Motor cables (not shown) within catheter tube 117, connector cable 160, and plug 170 may provide an electrical connection between the motor of the heart pump 110 and controller 130. During operation, controller 130 may be configured to receive measurements from one or more pressure sensors of the heart pump 110 through the sensor cables (e.g., optical fibers) and to control the electrical power delivered to the motor of the heart pump 110 through the motor cables. By controlling the power delivered to the motor of the heart pump 110, controller 130 may be operable to control the speed of the motor.

Various modifications can be made to cardiac support system 100 and one or more of its components. For instance, one or more additional sensors may be added to heart pump 100. In another example, a signal generator may be added to heart pump 100 to generate a signal indicative of the rotational speed of the motor of the heart pump 110. As another example, one or more components of cardiac support system 100 may be separated. For instance, display 140 may be incorporated into another device in communication with controller 130 (e.g., wirelessly or through one or more electrical cables).

A heart pump (e.g., heart pump 110) may include a pressure sensor (e.g., an optical pressure sensor) configured to detect a pressure within the aorta of a patient's heart when the heart pump is properly positioned. The pressure signal sensed by the pressure sensor may be used, at least in part, to determine correct positioning of the heart pump within the patient's heart and/or to determine a blood flow rate through the heart pump when in operation. For instance, the pressure signal may be used in combination with a motor current signal received from a motor current sensor (not shown) and a set of stored values to determine a flow rate of blood through the heart pump. The differential pressure across the aortic valve may also indirectly be determined based on the pressure signal measuring the pressure in the aorta and the set of stored values.

Conventional medical devices include a controller and a display, which may be used to monitor and/or control one or more aspects of the operation of the medical device and/or the patient's physiology. In some instances, at least some of the data received by the controller from the medical device may be provided to one or more additional computing devices via one or more networks. For example, the controller associated with a medical device may have limited memory and/or processing resources, which may restrict the ability of the controller to perform certain data analyses based on the received data. In such instances, the controller may provide at least some of the data received from the medical device to one or more computing devices (e.g., one or more network-based/cloud computing devices) for further processing.

The inventors have recognized and appreciated that sending data from a controller configured to control a medical device via a network interface may present security vulnerabilities to the controller. For instance, a nefarious actor may be able to connect to the network interface and control hardware or software executing on the controller to change one or more operating parameters of the medical device, thereby affecting the care of the patient. To avoid or mitigate such security vulnerabilities, a controller of a medical device may not include a network interface and/or a network interface of the controller may require user intervention to switch from a disabled state to an enabled state (e.g., to update the software executing on the controller). Although security risks may be mitigated in such scenarios, the controller may include limited processing and/or storage resources that may restrict the types of data analyses that can be performed on data received from the medical device.

The inventors have recognized and appreciated that it may be advantageous to have a controller architecture for a medical device that includes multiple computing devices communicatively coupled by a switchable data communication network (e.g., a Gb Ethernet network). By making the data communication network switchable, the computing device configured to control the medical device may be communicatively isolated from outside networks when, for example, the medical device is in operation, and may be coupled to additional processing resources, when, for example, the medical device is not in operation.

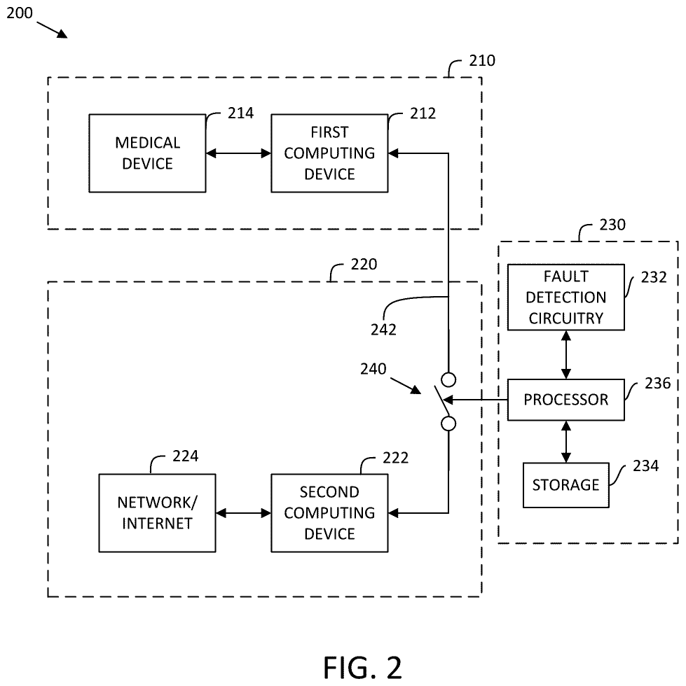

In some embodiments, the controller architecture may be implemented on a single substrate (e.g., a printed circuit board). FIG. 2 illustrates an example architecture 200 for a controller of a medical device in accordance with some embodiments. Architecture 200 includes a first computing subsystem 210, a second computing subsystem 220, and a third computing subsystem 230. The first computing subsystem 210 may include a first computing device 212 configured to control a medical device 214 (e.g., an implantable heart pump). Although only a single medical device 214 is shown, it should be appreciated that first computing device 212 may be configured to control multiple medical devices. In some embodiments, the first computing device 212 is a first system on a chip module (SOM). In some embodiments, the first SOM may have a smart mobility architecture (SMARC). The second computing subsystem 220 may include a second computing device 222. The second computing device 222 may include a network interface configured to connect to one or more network devices 224 (e.g., cloud computing resources, network connected displays, etc.). In some embodiments, second computing device 222 may be a second system on a chip module (SOM). In some embodiments, the second SOM may have a smart mobility architecture (SMARC).

In architecture 200, first computing device 212 and second computing device 222 may be communicatively coupled via a switchable data communication network 242. In some embodiments, switchable data communication network 242 is a high-speed data network (e.g., a Gb Ethernet network) that enables high speed data communication between the first computing device 212 and second computing device 222. As such, architecture 200 may utilize both an onboard or “internal” network (i.e., switchable data communication network 242) and an external network coupled between the second computing device 222 and network device(s) 224. Switchable data communication network 242 may include a switch 240 configured to be controlled by a processor 236 (e.g., a microprocessor). In some embodiments, processor 236 may be part of the third computing subsystem 230, which may also include fault detection circuitry 232 and storage 234. In some embodiments, processor 236 may be configured to control the state of one or more switches included in architecture 200, the one or more switches including switch 240. For example, processor 236 may be configured to control switch 240 to selectively enable or disable the switchable data communication network 242.

In some embodiments, processor 236 may be configured to selectively enable or disable the switchable data communication network 242 based on one or more conditions. For example, processor 236 may be configured to selectively enable or disable the switchable data communication network 242 based, at least in part, on an operating state of the medical device 214. For instance, when the medical device is operating, processor 236 may be configured to control switch 240 to selectively disable the switchable data communication network 242. Disabling the internal network 242 during operation of the medical device 214 may isolate the first computing subsystem 210, thereby reducing a security risk associated with having the first computing device 212 connected to an outside network via second computing device 222. In some embodiments, processor 236 may be configured to discover a current operating state of the medical device 214 by communicating with the first computing device 212. For instance, processor 236 may send a communication to the first computing device 212 to inquire about its current operating status and/or the functions that the first computing device is performing. In response, the first computing device 212 may send a communication to the processor 236 with the requested information. The processor 236 having received the information from the first computing device 212 can control the state of the switch 240 (and any other switches) accordingly to enable or disable switchable data communication network 242.

In some embodiments, processor 236 may be configured to selectively enable or disable the switchable data communication network 242 based, at least in part, on a state of a connection between the second computing device 222 and the at least one network device 224. For instance, processor 236 may be configured to selectively enable the switchable data communication network 242 when the second computing device 222 is disconnected from the at least one network device 224, and may be configured to selectively disable the switchable data communication network 242 when the second computing device is connected to the at least one network device 224. In some embodiments, processor 236 may be configured to selectively enable or disable switchable data communication network 242 based, at least in part, on a type of network device currently coupled the second computing device 222. For instance, processor 236 may be configured to selectively disable the switchable data communication network 242 when a WiFi enabled network device is coupled to the second computing device 222, but not disable the switchable data communication network 242 when an Ethernet-based network device is coupled to the second computing device 222. It should be appreciated that any suitable logic for determining when to selectively enable or disable switchable data communication network 242 based on one or more connected network devices may additionally or alternatively be used.

In some embodiments, processor 236 may be configured to selectively enable or disable the switchable data communication network 242 based, at least in part, on a task that a user wants to perform. For example, if a user wants to transfer data (e.g., one or more log files) from storage associated with the first computing device 212, the user may couple a storage device (e.g., external USB storage) to the network interface of the second computing device 222. The user may then interact with a user interface provided by the first computing device 212, the second computing device 222 or the processor 236 to initiate a transfer of the information. In response to the interaction, processor 236 may be configured to selectively enable switchable data communication network 242 to allow the information to be transferred from the storage associated with first computing device 212 to storage associated with second computing device 222. After the transfer has been completed, processor 236 may be configured to selectively disable switchable data communication network 242. Upon receiving the information from the first computing device 212, second computing device 222 may be configured to transfer the information to the storage device coupled to the network interface of the second computing device 222. In this way, the first computing device 212 may remain isolated from external networks unless necessary to perform a user-initiated task.

In another example, a user desiring to perform a software update may couple a storage device (e.g., external USB storage) to a network interface of the second computing device 222. The user may then interact with a user interface provided by the first computing device 212, the second computing device 222 or the processor 236 to initiate the software update (e.g., by providing security credentials, such as an administrative password via the user interface). In a first instance in which the software update relates to updating software on the second computing device 222, processor 236 may be configured to selectively disable (or keep disabled) switchable data communication network 242 because access to the first computing device 212 is not necessary to perform the software update. In a second instance in which the software update relates to updating software on the first computing device 212, processor 236 may be configured to selectively enable switchable data communication network 242 to allow software update information from the coupled storage device to be transferred to the first computing device 212. Following the software update, processor 236 may be configured to selectively disable switchable data communication network 242 to again isolate first computing device 212 from external network devices including the storage device coupled to the network interface of the second computing device 222.

In some embodiments, architecture 200 may include a plurality of switches and processor 236 may be configured to control each of the plurality of switches (e.g., by setting their state to enable or disable). In this way, processor 236 may be configured as a “master” processor capable of controlling and/or monitoring data communications between different components of architecture 200. When a fault or error condition is detected in a component of architecture 200, processor 236 may detect the fault or error condition and initiate an action (e.g., alert a user) based on the detected fault or error condition.

In some instances, processor 236 may itself experience a fault or reset condition that may not be detectable by processor 236. The inventors have recognized and appreciated the importance of detecting a failure or reset condition of processor 236 when it is configured in the manner described herein (e.g., as a master processor that controls and/or monitors onboard communication). In some embodiments, a fault or reset condition associated with processor 236 may be detected using fault detection circuitry 232. In some embodiments, fault detection circuitry 232 may be configured to output an alarm (e.g., a buzzer signal) when processor 236 experiences a fault or reset condition. For instance, processor 236 may be configured to suppress an alarm signal during normal operation. When the processor 236 experiences a fault or reset condition, the fault detection circuitry may enter a high-impedance mode causing the suppression of the alarm signal to be removed and the alarm signal to be output.

When a fault or reset condition for processor 236 occurs, some embodiments may be configured to keep all operations of architecture 200 in the same state that they were in prior to the fault or reset condition. For instance, if the medical device was in operation prior to the fault or reset condition, the medical device may continue to operate, whereas if the medical device was not operating when the fault or reset condition occurred, the medical device may remain off. In some embodiments, processor 236 may be configured to store the current state of each of a plurality of switches (or other outputs of components of architecture 200) in storage 234. For instance, storage 234 may include a plurality of registers. A plurality of state values may be cascaded through the plurality of registers, which may operate as flip-flops to store the state information. In this way, when a fault or reset condition associated with processor 236 occurs, the user may be alerted (e.g., via an alert signal output by the fault detection circuitry 232) and the current states of the switches in architecture 200 prior to the fault or reset condition may be stored. In some embodiments, the registers included in storage 234 may be readable. In such embodiments, when processor 236 becomes operational again (e.g., after a reset condition), processor 233 may be configured to send a message to read the state values in the registers to be able to automatically set the switches to their prior stored state (e.g., prior to the reset condition).

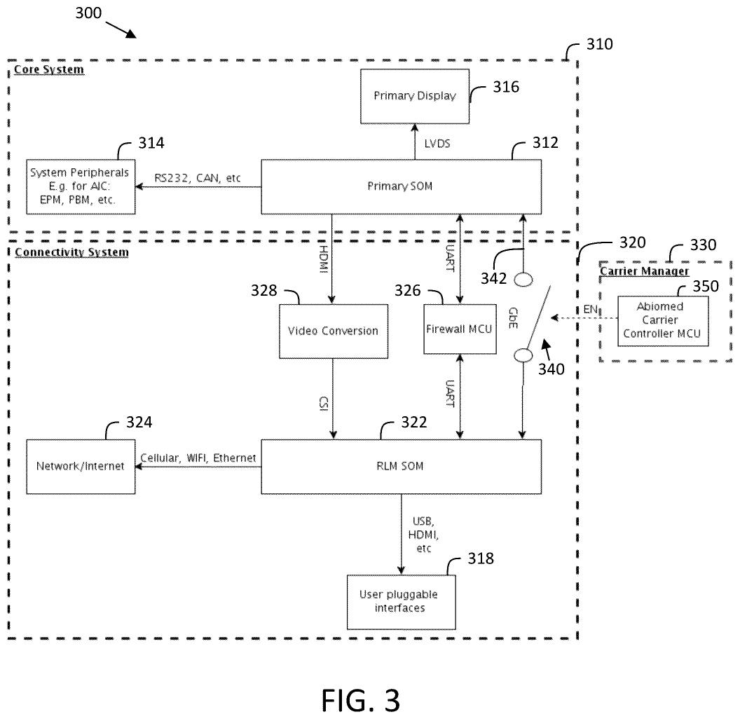

FIG. 3 illustrates an example architecture 300 for a controller of a medical device in accordance with some embodiments. Architecture 300 includes a core subsystem 310, a connectivity subsystem 320, and a manager subsystem 330. The core subsystem 310 may be configured to control operation of a medical device (e.g., an implantable heart pump) and receive data from the medical device during operation. As shown in FIG. 3, core subsystem 310 may include a primary computing device 312 (e.g., a primary system on a chip module (SOM)) and system peripherals 314 associated with the medical device. The primary computing device may be configured to send control signals to system peripherals 314. Core subsystem 310 may also include a primary display 316 (e.g., a display associated with an AIC). Primary display 316 may be configured to display data (e.g., medical device operation data and/or physiological data associated with a patient) provided by primary computing device 312.

Connectivity subsystem 320 may include a secondary computing device 322, also referred to herein as a remote link module (RLM) computing device (e.g., an RLM SOM). The secondary computing device 322 may include a network interface configured to connect to one or more network devices 324 (e.g., cloud computing resources, network connected displays, etc.). Any suitable communication protocol, examples of which include, but are not limited to cellular (e.g., LTE), WiFi, and Ethernet, may be used to connect the secondary computing device 322 to network device(s) 324.

As shown in FIG. 3, primary computing device 312 and secondary computing device 322 may be communicatively coupled via one or more data communication channels. One such data communication channel may be a switchable data communication network 342. In some embodiments, switchable data communication network 342 may be a high-speed data network (e.g., a Gb Ethernet network) that enables high speed data communication between the primary computing device 312 and the secondary computing device 322, thereby providing an onboard or “internal” network connection between the two computing devices of architecture 300. Switchable data communication network 342 may include a switch 340 configured to be controlled by a processor 350 (e.g., a microprocessor). In some embodiments, processor 350 may be part of the manager subsystem 330 configured to control and/or monitor the state of one or more switches included in architecture 300, the one or more switches including switch 340. For example, processor 350 may be configured to control switch 340 to selectively enable or disable the switchable data communication network 342.

As shown in FIG. 3, primary computing device 312 and secondary computing device 322 may additionally be communicatively coupled by a firewall component 326 (e.g., a hardware firewall component). Firewall component 326 may be configured to filter communication between the primary computing device 312 and the secondary computing device 322. In some embodiments, firewall component 326 enables bidirectional communication between the primary computing device 312 and the secondary computing device 322. In some embodiments, firewall component 326 is configured as a separate chip configured to perform the filtering of data communications passed through the chip. In some embodiments, firewall component 326 may include a switch (not shown), and processor 350 may be configured to enable or disable the switch to direct at least some communication between the primary computing device 312 and the secondary computing device 322 through the firewall component 326. In some embodiments, processor 350 may be configured to control the state of switch 340 and the switch associated with firewall component 326 based on one or more conditions. For instance, when primary computing device 312 is controlling a medical device to operate, processor 350 may control switch 340 to disable switchable data communication network and control the switch associated with firewall component 326 to enable some communication between primary computing device 312 and the secondary computing device 322 during operation of the medical device.

In some embodiments, primary computing device 312 and secondary computing device 322 may be further communicatively coupled via video conversion component 328. In some embodiments, video conversion component 328 may include a switch. The switch can either connect a first video stream received from the primary computing device 312 and having a first format (e.g., HDMI) to a network device (e.g., an external display) or convert the first video stream to a second video stream having a second format (e.g., camera serial interface (CSI), FPDisplay3, etc.) different from the first format, with the second video stream being provided to the secondary computing device 322.

In some embodiments, secondary computing device 322 may include at least one user pluggable interface 318 (e.g., a USB input, an HDMI input, etc.) to which a user may connect an external device. In some embodiments, user pluggable interface 318 may be a switchable interface including a switch having a state that may be controlled by processor 350. For example, user pluggable interface 318 may include a switchable USB interface, and processor 350 may be configured to selectively enable or disable one or more communication paths of the controller architecture 300 when a connector having a particular configuration is connected to the switchable USB interface. For example, processor 350 may be configured to detect when a service connector having a particular configuration is connected to the user pluggable interface 318. When the service connector is detected, processor 350 may be configured to couple a communication path between the pluggable interface and respective connectors on the primary computing device 312 and/or the secondary computing device 322 prior to booting the respective computing device, thereby enabling the respective computing device to boot from an external hard drive (e.g., a USB hard drive). When the service connector is not detected, processor 350 may be configured to couple a communication path between the pluggable interface and respective connectors on the primary computing device 312 and/or the secondary computing device 322 after booting the respective computing device, thereby enabling the respective computing device to boot from an internal memory device (e.g., onboard storage).

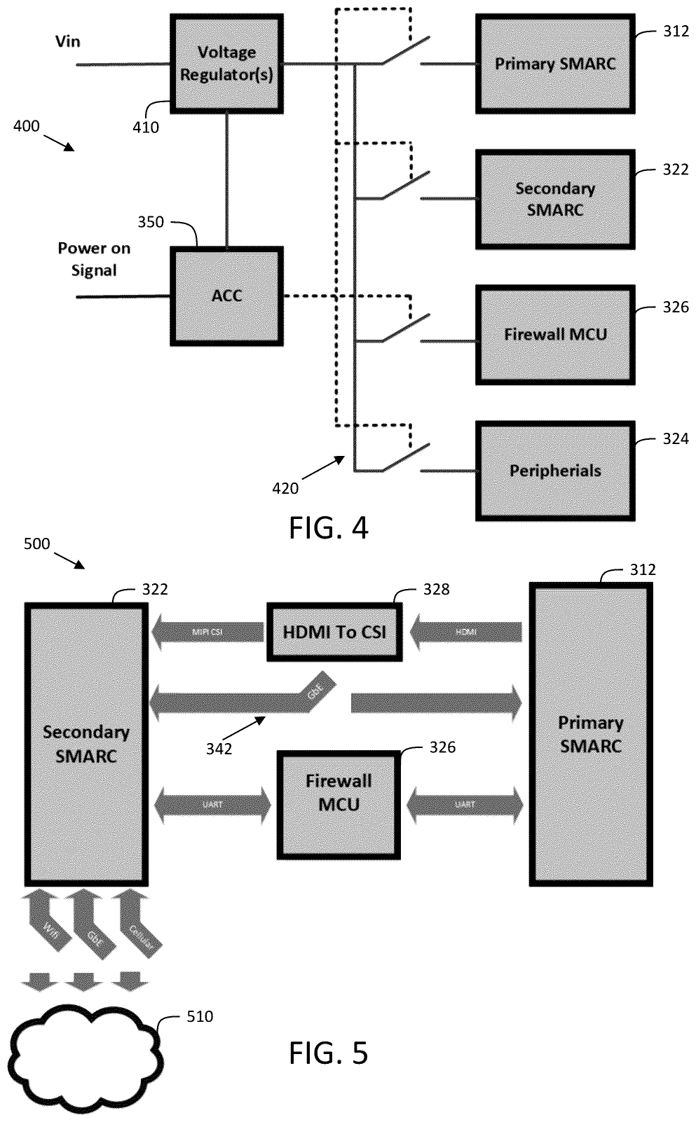

FIG. 4 schematically illustrates exemplary control pathways 400 for processor 350 shown in architecture 300, in accordance with some embodiments. In some embodiments, processor 350 may be configured to control power (e.g., an operating voltage) provided to different components of architecture 300. For instance, when power is provided through a Vin connection, voltage regulator 410 (e.g., a 3.3V voltage regulator) may provide power to the processor 350. In response to receiving a power on signal, processor 350 may be configured to enable the switches 420 (e.g., load switches) coupled to one or more components of architecture 300. In some embodiments, the switches 420 may have settable current limits and/or other safety features (inrush control). In some embodiments, processor 350 may be configured to enable/disable particular switches 420 in a particular sequence as required for a particular computing device (e.g., primary computing device 312, secondary computing device 322) or peripheral device (e.g., peripheral device 324).

FIG. 5 schematically illustrates a data communication architecture 500 communicatively coupling a primary computing device 312 (e.g., a first system on a chip module) and a secondary computing device 322 (e.g., a second system on a chip module). As described in connection with architecture 300 in FIG. 3, primary computing device 312 and secondary computing device may be communicatively coupled via a switchable data communication network 342 which, when enabled, may permit high-speed data transfer between the primary and secondary computing devices. In some embodiments, the switchable data communication network is a Gb Ethernet network. As described in connection with architecture 300, primary computing device 312 and secondary computing device 322 may further be communicatively coupled via firewall component 326 and/or video conversion component 328. The functions of these communication paths are described herein in connection with architecture 300 shown in FIG. 3. In some embodiments, some or all of the communication paths shown in data communication architecture 500 may be enabled or disabled by a processor (e.g., processor 350 of architecture 300) based on one or more conditions. By selectively enabling/disabling various communication paths, the “patient side” primary computing device 312, which may be configured to control one or more medical devices may be isolated from the “remote side” secondary computing device 322, which may be configured to interface with one or more network devices (e.g., cloud resources 510). Among other things, such isolation may permit different classes of software to be installed and executed on the primary and secondary computing devices. For instance, class 3 software subjected to more stringent safety regulations may be installed and executed on the primary computing device 312, whereas class 1 software subjected to less stringent safety regulations may be installed and executed on the secondary computing device 322. Additionally, by isolating the secondary computing device 322 from the controller used to control operation of the medical device, the software executing on the secondary computing device 322 may be updated more frequently and without having to recertify the software according to particular safety requirements.

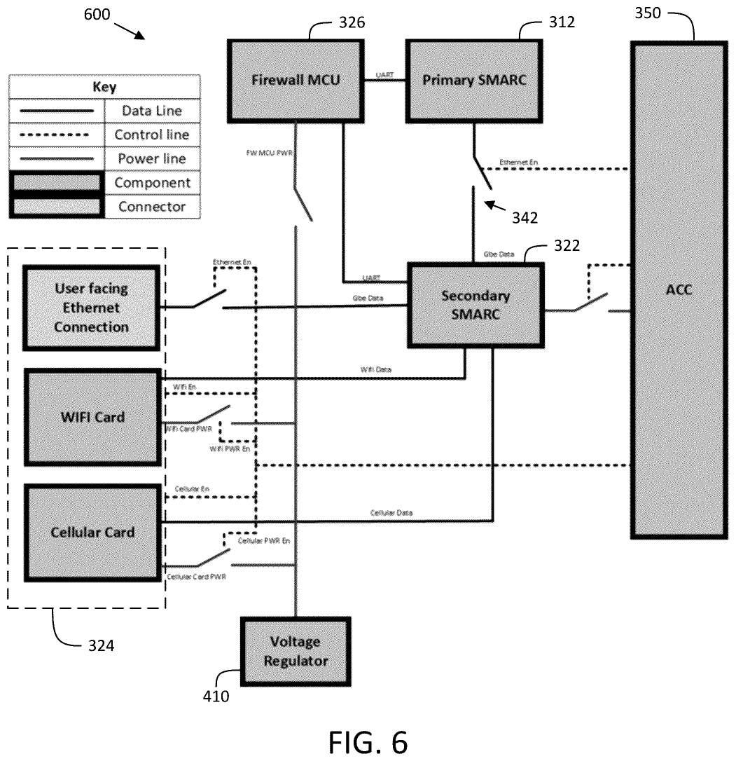

FIG. 6 illustrates an example architecture 600 for a controller of a medical device in accordance with some embodiments. As shown in FIG. 6, architecture 600 includes several of the same components shown and described in connection with architecture 300 shown in FIG. 3, including primary computing device 312, secondary computing device 322, firewall component 328 and processor 350. In architecture 600, control and power lines (e.g., as described in connection with control pathways 400 of FIG. 4) are shown. As described herein, processor 350 may be configured to control a state of a plurality of switches in architecture 600 to selectively enable or disable associated components, thereby controlling how data communication is provided between the different components.

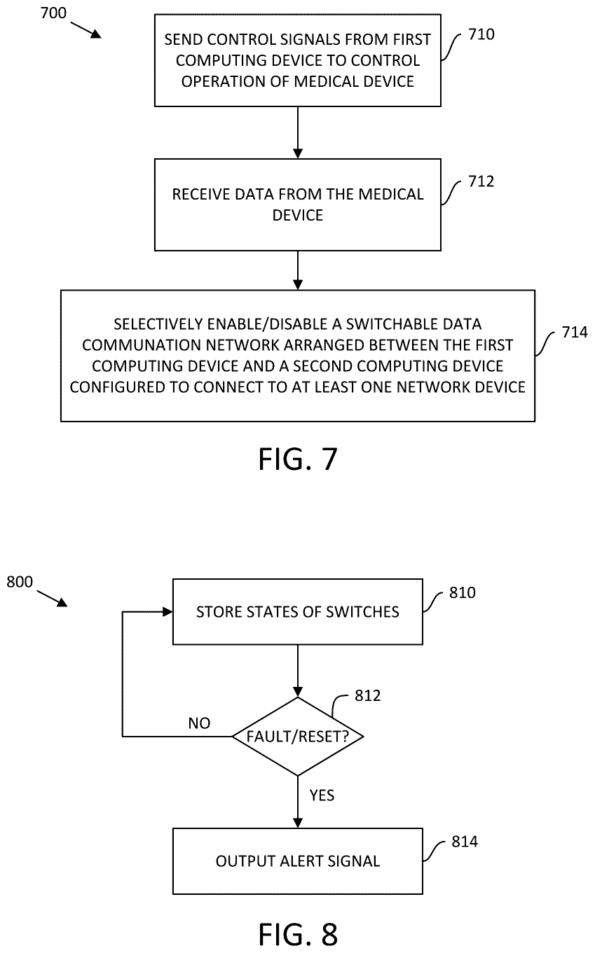

FIG. 7 is a flowchart of a process 700 for selectively enabling or disabling an onboard switchable data communication network for a controller of a medical device in accordance with some embodiments of the present disclosure. In act 710, one or more control signals may be sent from a first computing device (e.g., a first SOM) to control an operation of a medical device (e.g., an implantable heart pump, an extracorporeal membrane oxygenation (ECMO) system, etc.). Process 700 then proceeds to act 712, where data is received by the first computing device from the medical device being controlled. For instance, the received data may include data describing an operation of the medical device (e.g., a pump speed, a motor current, a temperature of the device) and/or physiological information associated with a patient using the medical device (e.g., a heart rate, one or more sensed pressures, etc.). At least some of the data received by the first computing device may be provided as one or more graphs and/or values displayed on a monitor associated with the first computing device. Process 700 may then proceed to act 714, where a switchable data communication network (e.g., a high-speed Ethernet network) arranged between the first computing device and a second computing device configured to connect to at least one network device is selectively enabled or disabled based on one or more conditions. For instance, as described herein, the switchable data communication network may be enabled or disabled based, at least in part, on an operating condition of the medical device, a network connection status of one or more network interfaces of the secondary computing device, whether a connector having a particular configuration is connected to a user pluggable interface of the second computing device, a combination of these conditions, or other conditions. When the switchable data communication network is enabled, at least some of the data received by the first computing device may be provided to the second computing device via the switchable data communication network.

FIG. 8 is a flowchart of a process 800 for monitoring for a fault or reset state of a manager processor (e.g., processor 236 in architecture 200, processor 350 in architecture 300), in accordance with some embodiments. As described herein, a controller architecture designed in accordance with some embodiments may include a plurality of switches having different states controlled by a processor. Process 800 may begin in act 810, where a plurality of current states of the plurality of switches are stored (e.g., using registers included in storage 234 associated with processor 236 in architecture 200). Process 800 may then proceed to act 812, where it is determined whether a fault or reset condition of the manager processor has occurred. Examples of detecting whether a fault or reset condition are described herein. If it is determined in act 812 that a fault or reset condition has not occurred, process 800 may return to act 810, where the plurality of current states of the switches is updated, and the process may repeat until it is determined in act 812 that a fault or reset condition of the manager processor is detected. When a fault or reset condition is detected in act 812, process 800 may proceed to act 814, where an alert signal is output. For instance, as described herein, in some embodiments an alert signal may be suppressed by the manager processor during normal operation (e.g., while a fault or reset condition is not detected in act 812), and the suppressed alert signal may be output in act 814 when a fault or reset condition is detected in act 812.

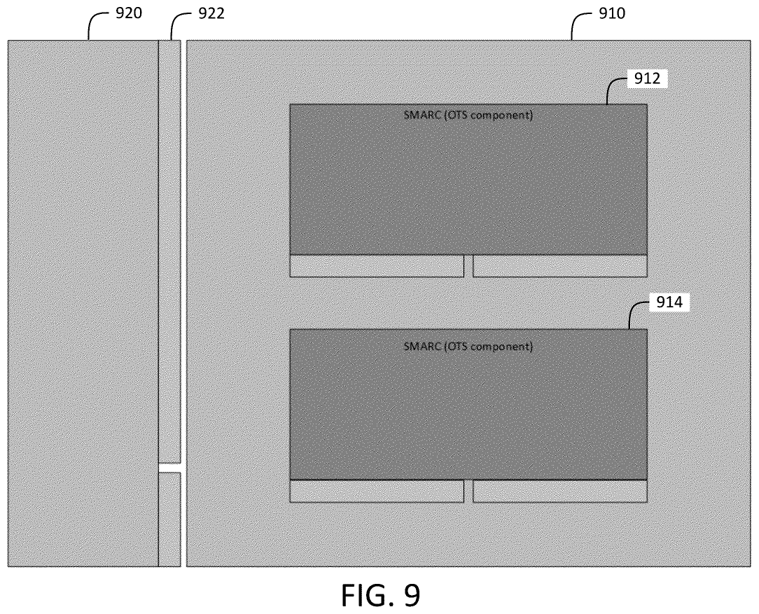

As described herein, a controller for a medical device according to some embodiments may including multiple computing devices integrated on a single printed circuit board. FIG. 9 illustrates an example of a printed circuit board 910 that includes a first computing device 912 and a second computing device 914. In some embodiments, printed circuit board 910 includes a standard edge connector that may be coupled to an expansion board 920. When expansion boards having different pin configurations are coupled to the standard edge connector, additional functionality may be provided to the control architecture. For example, in some embodiments, the standard edge connector of the printed circuit board 910 may be configured to couple to expansion boards for different processor architectures, which may enable evaluation and/or testing of the printed circuit board 910 without having to have separate evaluation platforms. In some embodiments, expansion board 920, when coupled to printed circuit board 910 may provide additional processing and/or storage resources not resident on printed circuit board 910, which may facilitate the use of advanced data analysis algorithms (e.g. machine learning algorithms) to process the data received from a medical device.

Having thus described several aspects and embodiments of the technology set forth in the disclosure, it is to be appreciated that various alterations, modification, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The above-described embodiments of the present technology can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as a controller that controls the above-described function. A controller can be implemented in numerous ways, such as with dedicated hardware, or with general purpose hardware (e.g., one or more processor) that is programmed using microcode or software to perform the functions recited above, and may be implemented in a combination of ways when the controller corresponds to multiple components of a system.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

1. A controller for a medical device, the controller comprising:
a first computing device configured to control the medical device;
a second computing device configured to connect to at least one network device, wherein the first computing device and the second computing device are coupled via a switchable data communication network; and
a processor configured to selectively enable or disable the switchable data communication network.

2. The controller of claim 1, further comprising:
a firewall component arranged between the first computing device and the second computing device, the firewall component configured to filter communication between the first computing device and the second computing device.

3. The controller of claim 2, further comprising a first switch, wherein the processor is further configured enable or disable the first switch to direct at least some communication between the first computing device and the second computing device through the firewall component.

4. The controller of claim 1, further comprising:
a video conversion component arranged between the first computing device and the second computing device, the video conversion component configured to convert a first video stream received from the first computing device and having first format to a second video stream having a second format.

5. The controller of claim 4, further comprising a second switch, wherein
the processor is further configured to control a state of the second switch,
when the second switch is in a first state, the first video stream is provided to an external device, and
when the second switch is in a second state, the first video stream is converted to the second video stream, the second video stream being provided as input to the second computing device.

6. (canceled)

7. The controller of claim 1, wherein the second computing device includes a switchable user interface.

8. The controller of claim 7, wherein the processor is further configured to:
determine when a connector having a particular configuration is connected to the switchable user interface; and
disable the switchable user interface when it is determined that a connector having the particular configuration is connected to the switchable interface.

9. The controller of claim 1, wherein the first computing device is a first system on chip module (SOM) and the second computing device is a second SOM.

10. (canceled)

11. The controller of claim 1, wherein the processor is configured to selectively enable the switchable data communication network when the second computing device is disconnected from the at least one network device.

12. The controller of claim 1, wherein the processor is configured to selectively disable the switchable data communication network when the second computing device is connected to the at least one network device.

13. The controller of claim 1, wherein the processor is configured to selectively enable or disable the switchable data communication network based, at least in part, on an operating state of the medical device.

14. The controller of claim 13, wherein the processor is configured to selectively disable the switchable data communication network when the medical device is operating.

15. The controller of claim 1, further comprising a storage device in communication with the processor, wherein the processor is further configured to store a plurality of states associated with switches in the controller.

16. The controller of claim 1, wherein the processor is further configured to selectively provide power to one or more components of the controller.

17. The controller of claim 1, further comprising fault detection circuitry arranged to detect a fault or a reset condition of the processor.

18. The controller of claim 17, wherein the fault detection circuitry is configured to output an alarm signal in response to detecting a fault or a reset condition of the processor.

19. The controller of claim 18, wherein the processor is configured to suppress output of the alarm signal unless a fault or a reset condition of the processor occurs.

20. The controller of claim 1, wherein the first computing device, the second computing device and the processor are arranged on a printed circuit board.

21. A method of controlling a medical device, the method comprising:
sending control signals from a first computing device to control an operation of the medical device;
receiving, by the first computing device, data from the medical device; and
selectively enabling or disabling, by a processor, a switchable data communication network, the switchable data communication network arranged to enable communication between the first computing device and a second computing device configured to connect to at least one network device.

22-37. (canceled)

38. A printed circuit board, comprising:
a first system on a chip module (SOM) configured to control a medical device;
a second SOM configured to connect to at least one network device; and
a switchable data communication network coupled between the first SOM and the second SOM.
